(19) United States
(12) Patent Application Publication
(10) Pub. No.: US 2017/0253575 A1
(43) Pub. Date: Sep. 7, 2017

(54) SOLID ACID CATALYST AND METHOD FOR PRODUCING OXIDE

(71) Applicant: DAICEL CORPORATION, Osaka-shi, Osaka (JP)

(72) Inventors: Takamasa SUZUKI, Himeji-shi (JP); Ichiro TAKASE, Himeji-shi (JP)

(73) Assignee: DAICEL CORPORATION, Osaka-shi, Osaka (JP)

(21) Appl. No.: 15/507,000

(22) PCT Filed: Jul. 31, 2015
(86) PCT No.: PCT/JP2015/071827

§ 371 Date: Feb. 27, 2017

(30) Foreign Application Priority Data
Aug. 29, 2014 (JP) 2014-175314

(51) Int. Cl. C07D 311/76; B01J 31/10; B01J 35/00; B01J 35/02; C07C 51/34; B01J 37/02; B01J 37/00; B01J 37/30; C07C 29/48; C07C 45/40; B01J 23/889; B01J 37/04

(52) U.S. Cl. CPC C07D 311/76; B01J 23/8892; B01J 31/10; B01J 35/0006; B01J 35/02; B01J 35/026; B01J 37/04; B01J 37/0221; B01J 37/0219; B01J 37/009; B01J 37/0236; B01J 37/30; C07C 29/48; C07C 45/40; C07C 51/34; B01J 2231/70; B01J 2531/005; B01J 2531/008; C07C 2601/14; C07C 2601/16

(57) ABSTRACT

Provided is a solid acid catalyst for use in oxidation of a substrate in the coexistence of oxygen and ozone (solid acid catalyst for oxygen-ozone-coexisting oxidation). The solid acid catalyst enables oxidation of the substrate with a high conversion. This solid acid catalyst for oxygen-ozone-coexisting oxidation is a solid acid catalyst for use in an oxidation reaction to oxidize a substrate (A) in the coexistence of oxygen and ozone. The solid acid catalyst includes a transition metal in the form of an elementary substance, a compound, or an ion, and a support supporting the transition metal. The support includes, at least in its surface, a strong acid or super strong acid having a Hammett acidity function (H₀) of −9 or less. The support is preferably a pellet or particle made of a fluorinated sulfonic acid resin, or a support including a solid and a layer of a fluorinated sulfonic acid resin disposed on the solid.

### TECHNICAL FIELD

The present invention relates to a solid acid catalyst for oxygen-ozone-coexisting oxidation reactions, which catalyst is for use in oxidation of substrates in the coexistence of oxygen and ozone. The present invention is also relates to a method for producing an oxide (oxidation product) using the solid acid catalyst. This application claims priority to Japanese Patent Application No. 2014-175314, filed Aug. 29, 2014 to Japan, the entire contents of which are incorporated herein by reference.

### BACKGROUND ART

Oxidation reactions are one of most basic reactions in the organic chemical industry, for which various oxidation methods have been developed. Patent Literature (PTL) 1 describes an oxidation method by which a compound capable of forming a radical is oxidized typically with molecular oxygen by the catalysis of a lipid-soluble imide compound.

With this method, a wide variety of organic substrates can be oxidized to give corresponding oxides. Disadvantageously, however, this method is not always considered to be an industrially satisfactory method in points typically of substrate conversion and yield of the target compound.

### CITATION LIST

### Patent Literature

PTL 1: Japanese Unexamined Patent Application Publication (JP-A) No. 2002-331242

### SUMMARY OF INVENTION

### Technical Problem

Accordingly, the present invention has an object to provide a solid acid catalyst for oxygen-ozone-coexisting oxidation reactions, which is for use in oxidation of a substrate in the coexistence of oxygen and ozone and which enables oxidation of the substrate with a high conversion.

The present invention has another object to provide a method for producing an oxide (such as an oxygen-containing compound) from a substrate in a high yield.

### Solution to Problem

After intensive investigations to achieve the objects, the inventors of the present invention found that a substrate, when oxidized in the presence of a specific solid acid catalyst and in the coexistence of oxygen and ozone, can be oxidized with a high conversion and gives a corresponding oxide (oxidation product) (such as an oxygen-containing compound) in a high yield. The present invention has been made based on these findings.

Specifically, the present invention relates to the followings.

(1) The present invention relates to a solid acid catalyst for use in an oxidation reaction to oxidize a substrate (A) in the coexistence of oxygen and ozone (solid acid catalyst for oxygen-ozone-coexisting oxidation). The solid acid catalyst includes a transition metal in the form of an elementary substance, a compound, or an ion, and a support supporting the transition metal. The support includes or bears, at least in its surface, a strong acid or super strong acid having a Hammett acidity function (H₀) of −9 or less. The substrate (A) includes a compound selected from the group consisting of (A1) compounds containing a heteroatom and a carbon-hydrogen bond at a position adjacent to the heteroatom, (A2) compounds containing a carbon-heteroatom double bond, (A3) compounds containing a methine carbon atom, (A4) compounds containing an unsaturated bond and a carbon-hydrogen bond at a position adjacent to the unsaturated bond, (A5) alicyclic compounds, (A6) conjugated compounds, (A7) amine compounds, (A8) aromatic compounds, (A9) straight chain alkanes, and (A10) olefins.

(2) The present invention also relates to a solid acid catalyst for use in an oxidation reaction to oxidize a substrate (A) in the coexistence of oxygen and ozone (solid acid catalyst for oxygen-ozone-coexisting oxidation). The solid acid catalyst includes a transition metal in the form of an elementary substance, a compound, or an ion, and a support supporting the transition metal. The support includes or bears, at least in its surface, at least one strong acid or super strong acid selected from the group consisting of sulfuric acid, sulfated metal oxides, noble metals/sulfated metal oxides, metal oxide super strong acids, and fluorinated sulfonic acid resins. The substrate (A) includes a compound selected from the group consisting of (A1) compounds containing a heteroatom and a carbon-hydrogen bond at a position adjacent to the heteroatom, (A2) compounds containing a carbon-heteroatom double bond, (A3) compounds containing a methine carbon atom, (A4) compounds containing an unsaturated bond and a carbon-hydrogen bond at a position adjacent to the unsaturated bond, (A5) alicyclic compounds, (A6) conjugated compounds, (A7) amine compounds, (A8) aromatic compounds, (A9) straight chain alkanes, and (A10) olefins.

(3) In the solid acid catalyst according to one of (1) to (2), the compounds (A1) containing a heteroatom and a carbon-hydrogen bond at a position adjacent to the heteroatom may be selected from (A1-1) primary or secondary alcohols, and primary or secondary thiols; (A1-2) ethers containing a carbon-hydrogen bond at a position adjacent to oxygen, and sulfides containing a carbon-hydrogen bond at a position adjacent to sulfur; and (A1-3) acetals (including hemiacetals) containing a carbon-hydrogen bond at a position adjacent to oxygen, and thioacetals (including thiohemiacetals) containing a carbon-hydrogen bond at a position adjacent to sulfur.

(4) In the solid acid catalyst according to any one of (1) to (3), the compounds (A2) containing a carbon-heteroatom double bond may be selected from (A2-1) carbonyl-containing compounds, (A2-2) thiocarbonyl-containing compounds, and (A2-3) imines.

(5) In the solid acid catalyst according to any one of (1) to (4), the compounds (A3) containing a methine carbon atom may be selected from (A3-1) cyclic compounds containing a methine group (namely, a methine carbon-hydrogen bond) as a ring-constituting unit, and (A3-2) chain compounds containing a methine carbon atom.

(6) In the solid acid catalyst according to any one of (1) to (5), the compounds (A4) containing an unsaturated bond and a carbon-hydrogen bond at a position adjacent to the unsaturated bond may be selected from (A4-1) aromatic compounds containing an aromatic ring and a methyl or methylene group at a position adjacent to the aromatic ring (at the so-called “benzylic position”), and (A4-2) non-aromatic compounds containing an unsaturated bond and a methyl or methylene group at a position adjacent to the unsaturated bond.

(7) In the solid acid catalyst according to any one of (1) to (6), the alicyclic compounds (A5) (i.e., non-aromatic cyclic hydrocarbons) may be selected from (A5-1) cycloalkanes and (A5-2) cycloalkenes.

(8) In the solid acid catalyst according to any one of (1) to (7), the conjugated compounds (A6) may be selected from (A6-1) conjugated dienes, (A6-2) α,β-unsaturated nitriles, and (A6-3) α,β-unsaturated carboxylic acids and derivatives thereof.

(9) In the solid acid catalyst according to any one of (1) to (8), the amine compounds (A7) may be selected from primary or secondary amines.

(10) In the solid acid catalyst according to any one of (1) to (9), the aromatic compounds (A8) may be selected from aromatic hydrocarbons containing at least one benzene ring, and fused polycyclic aromatic hydrocarbons containing at least two benzene rings fused to each other.

(11) In the solid acid catalyst according to any one of (1) to (10), the straight chain alkanes (A9) may be selected from C₁-C₃₀ straight chain alkanes.

(12) In the solid acid catalyst according to any one of (1) to (11), the olefins (A10) may be selected from chain olefins and cyclic olefins.

(13) In the solid acid catalyst according to any one of (1) to (12), the substrate (A) may include a hydrocarbon.

(14) In the solid acid catalyst according to (13), the hydrocarbon may be at least one selected from the group consisting of aliphatic hydrocarbons, alicyclic hydrocarbons, and aromatic hydrocarbons.

(15) In the solid acid catalyst according to any one of (1) to (14), the support may be selected from a support including a fluorinated sulfonic acid resin in the form of a pellet or a particle, and a support including a solid and a fluorinated sulfonic acid resin in the form of a layer disposed on the solid.

(16) In the solid acid catalyst according to any one of (1) to (15), the transition metal may be at least one selected from the group consisting of cobalt, manganese, vanadium, iron, zirconium, tungsten, and molybdenum.

(17) In the solid acid catalyst according to any one of (1) to (16), the transition metal may be at least one selected from the group consisting of cobalt, manganese, vanadium, iron, and zirconium.

(18) In the solid acid catalyst according to any one of (1) to (17), the transition metal may be at least one selected from the group consisting of cobalt and manganese.

(19) The solid acid catalyst according to any one of (1) to (18) may be pellet-like, film-like, or tubular in shape.

(20) The present invention also relates to a method for producing an oxide. The method includes oxidizing a substrate (A) in the presence of the solid acid catalyst according to any one of (1) to (19) and in the coexistence of oxygen and ozone. The substrate (A) includes a compound selected from the group consisting of (A1) compounds containing a heteroatom and a carbon-hydrogen bond at a position adjacent to the heteroatom, (A2) compounds containing a carbon-heteroatom double bond, (A3) compounds containing a methine carbon atom, (A4) compounds containing an unsaturated bond and a carbon-hydrogen bond at a position adjacent to the unsaturated bond, (A5) alicyclic compounds, (A6) conjugated compounds, (A7) amine compounds, (A8) aromatic compounds, (A9) straight chain alkanes, and (A10) olefins.

(21) In the method according to (20) for producing an oxide, the substrate may be oxidized further in the presence of an imide compound having a cyclic imide skeleton.

### Advantageous Effects of Invention

According to the present invention, a substrate is oxidized in the presence of the specific solid acid catalyst and in the coexistence of oxygen and ozone. This configuration offers significantly higher oxidation rates and enables oxidation of a wide variety of substrates with high conversions. In addition, the configuration gives an oxide (such as an oxygen-containing compound) in a high yield even when the substrate is selected from hydrocarbons, in particular, selected from straight chain alkanes.

### DESCRIPTION OF EMBODIMENTS

Reaction Substrate

An organic compound or compounds (organic substrate or substrates) for use as the reaction substrate (A) in the present invention are preferably selected from compounds that are capable of forming a stable radical. Such compounds may be selected from (A1) compounds containing a heteroatom and a carbon-hydrogen bond at a position adjacent to the heteroatom, (A2) compounds containing a carbon-heteroatom double bond, (A3) compounds containing a methine carbon atom, (A4) compounds containing an unsaturated bond and a carbon-hydrogen bond at a position adjacent to the unsaturated bond, (A5) alicyclic compounds, (A6) conjugated compounds, (A7) amine compounds, (A8) aromatic compounds, (A9) straight chain alkanes, and (A10) olefins.

These compounds may each have one or more of various substituents within ranges not adversely affecting the reaction. Non-limiting examples of the substituents include halogens, hydroxy, mercapto, oxo, substituted oxys (such as alkoxys, aryloxys, and acyloxys), substituted thios, carboxy, substituted oxycarbonyls, substituted or unsubstituted carbamoyls, cyano, nitro, substituted or unsubstituted aminos, sulfo, alkyls, alkenyls, alkynyls, alicyclic hydrocarbon groups, aromatic hydrocarbon groups, and heterocyclic groups.

Non-limiting examples of the compounds (A1) containing a heteroatom and a carbon-hydrogen bond at a position adjacent to the heteroatom include (A1-1) primary or secondary alcohols, and primary or secondary thiols; (A1-2) ethers containing a carbon-hydrogen bond at a position adjacent to oxygen, and sulfides containing a carbon-hydrogen bond at a position adjacent to sulfur; and (A1-3) acetals (including hemiacetals) containing a carbon-hydrogen bond at a position adjacent to oxygen, and thioacetals (including thiohemiacetals) containing a carbon-hydrogen bond at a position adjacent to sulfur.

The primary or secondary alcohols in the category (A1-1) include a wide variety of alcohols. The alcohols may be any of monohydric, dihydric, and polyhydric alcohols.

Representative, but non-limiting examples of the primary alcohols include saturated or unsaturated aliphatic primary alcohols containing 1 to about 30 (preferably 1 to 20, and particularly preferably 1 to 15) carbon atoms, such as methanol, ethanol, 1-propanol, 1-butanol, 1-pentanol, 1-hexanol, 1-octanol, 1-decanol, 2-buten-1-ol, ethylene glycol, trimethylene glycol, hexamethylene glycol, and pentaerythritol; saturated or unsaturated alicyclic primary alcohols such as cyclopentylmethyl alcohol, cyclohexylmethyl alcohol, and 2-cyclohexylethyl alcohol; aromatic primary alcohols such as benzyl alcohol, 2-phenylethyl alcohol, 3-phenylpropyl alcohol, and cinnamic alcohol; and heterocyclic alcohols such as 2-hydroxymethylpyridine.

Representative, but non-limiting examples of the secondary alcohols include saturated or unsaturated aliphatic secondary alcohols containing 3 to about 30 (preferably 3 to 20, and particularly preferably 3 to 15) carbon atoms, such as 2-propanol, s-butyl alcohol, 2-pentanol, 2-octanol, and 2-penten-4-ol, as well as vicinal diols such as 1,2-propanediol, 2,3-butanediol, and 2,3-pentanediol; secondary alcohols containing an aliphatic hydrocarbon group and an alicyclic hydrocarbon group (such as cycloalkyl) each bonded to a carbon atom to which a hydroxy group is bonded, such as 1-cyclopentylethanol and 1-cyclohexylethanol; saturated or unsaturated alicyclic secondary alcohols (including bridged secondary alcohols) containing 3 to about 30 members (preferably 3 to 15 members, particularly preferably 5 to 15 members, and most preferably 5 to 8 members), such as cyclopentanol, cyclohexanol, cyclooctanol, cyclododecanol, 2-cyclohexen-1-ol, 2-adamantanol, 2-adamantanols containing one to four hydroxy groups at a bridgehead position or positions, and 2-adamantanols containing an oxo group on the adamantane ring; aromatic secondary alcohols such as 1-phenylethanol; and heterocyclic secondary alcohols such as 1-(2-pyridyl)ethanol.

Representative, but non-limiting examples of the alcohols also include alcohols containing a bridged hydrocarbon group, such as 1-adamantanemethanol, α-methyl-1-adamantanemethanol, 3-hydroxy-α-methyl-1-adamantanemethanol, 3-carboxy-α-methyl-1-adamantanemethanol, α-methyl-3a-perhydroindenemethanol, α-methyl-4a-decahydronaphthalenemethanol, α-methyl-4a-perhydrofluorenemethanol, α-methyl-2-tricyclo[5.2.1.0^{2,6}]decanemethanol, and α-methyl-1-norbornanemethanol, of which compounds containing a bridged hydrocarbon group bonded to a carbon atom to which a hydroxy group is bonded are typified.

Non-limiting examples of the primary or secondary thiols in the category (A1-1) include thiols corresponding to the primary or secondary alcohols.

Non-limiting examples of the ethers containing a carbon-hydrogen bond at a position adjacent to oxygen in the category (A1-2) include aliphatic ethers such as dimethyl ether, diethyl ether, dipropyl ether, diisopropyl ether, dibutyl ether, and diallyl ether; aromatic ethers such as anisole, phenetole, dibenzyl ether, and phenyl benzyl ether; and cyclic ethers (to which an aromatic ring or a non-aromatic ring may be fused), such as dihydrofuran, tetrahydrofuran, pyran, dihydropyran, tetrahydropyran, morpholine, chroman, and isochroman.

Non-limiting examples of the sulfides containing a carbon-hydrogen bond at a position adjacent to sulfur in the category (A1-2) include sulfides corresponding to the ethers containing a carbon-hydrogen bond at a position adjacent to oxygen.

The acetals containing a carbon-hydrogen bond at a position adjacent to oxygen in the category (A1-3) are exemplified by acetals each derived from an aldehyde typically with an alcohol or an acid anhydride, and such acetals include cyclic acetals and non-cyclic acetals. Non-limiting examples of the aldehyde include aliphatic aldehydes such as formaldehyde, acetaldehyde, propionaldehyde, butyraldehyde, and isobutyraldehyde; alicyclic aldehydes such as cyclopentanecarbaldehyde and cyclohexanecarbaldehyde; and aromatic aldehydes such as benzaldehyde and phenylacetaldehyde. Non-limiting examples of the alcohol include monohydric alcohols such as methanol, ethanol, 1-propanol, 1-butanol, and benzyl alcohol; and dihydric alcohols such as ethylene glycol, propylene glycol, 1,3-propanediol, and 2,2-dibromo-1,3-propanediol. Representative, but non-limiting examples of the acetals include 1,3-dioxolane compounds such as 1,3-dioxolane, 2-methyl-1,3-dioxolane, and 2-ethyl-1,3-dioxolane; 1,3-dioxane compounds such as 2-methyl-1,3-dioxane; and dialkyl acetal compounds such as acetaldehyde dimethyl acetal.

Non-limiting examples of the thioacetals containing a carbon-hydrogen bond at a position adjacent to sulfur in the category (A1-3) include thioacetals corresponding to the acetals containing a carbon-hydrogen bond at a position adjacent to oxygen.

Non-limiting examples of the compounds (A2) containing a carbon-heteroatom double bond include (A2-1) carbonyl-containing compounds, (A2-2) thiocarbonyl-containing compounds, and (A2-3) imines. The carbonyl-containing compounds (A2-1) include ketones and aldehydes and are exemplified by, but not limited to, chain ketones such as acetone, methyl ethyl ketone, methyl isobutyl ketone, 3-pentanone, methyl vinyl ketone, methyl cyclohexyl ketone, and acetophenone; cyclic ketones such as cyclopentanone, cyclohexanone, 4-methylcyclohexanone, isophorone, cyclodecanone, cyclododecanone, 1,4-cyclooctanedione, 2,2-bis(4-oxocyclohexyl)propane, and 2-adamantanone; 1,2-dicarbonyl compounds such as biacetyl (2,3-butanedione), bibenzoyl (i.e., benzil), acetylbenzoyl, and cyclohexane-1,2-dione, of which α-diketones are typified; α-keto-alcohols such as acetoin and benzoin; aliphatic aldehydes such as acetaldehyde, propionaldehyde, butanal, hexanal, succinaldehyde, glutaraldehyde, and adipaldehyde; alicyclic aldehydes such as cyclohexylaldehyde, citral, and citronellal; aromatic aldehydes such as benzaldehyde, carboxybenzaldehyde, nitrobenzaldehyde, cinnamaldehyde, salicylaldehyde, anisaldehyde, phthalaldehyde, isophthalaldehyde, and terephthalaldehyde; and heterocyclic aldehydes such as furfural and nicotinaldehyde.

Non-limiting examples of the thiocarbonyl-containing compounds (A2-2) include thiocarbonyl-containing compounds corresponding to the carbonyl-containing compounds (A2-1).

Examples of the imines (A2-3) include imines (including oximes and hydrazones) derived from any of the carbonyl-containing compounds (A2-1) with any of ammonia and amines. Non-limiting examples of the amines include amines such as methylamine, ethylamine, propylamine, butylamine, hexylamine, benzylamine, cyclohexylamine, and aniline; hydroxyamines such as hydroxylamine and O-methylhydroxyamine; and hydrazines such as hydrazine, methylhydrazine, and phenylhydrazine.

The compounds (A3) containing a methine carbon atom include (A3-1) cyclic compounds containing a methine group (namely, a methine carbon-hydrogen bond) as a ring-constituting unit; and (A3-2) chain compounds containing a methine carbon atom.

The cyclic compounds (A3-1) include (A3-1a) bridged compounds containing at least one methine group; and (A3-1b) non-aromatic cyclic compounds (such as alicyclic hydrocarbons) containing a ring and a hydrocarbon group bonded to the ring. The bridged compounds also include compounds containing two rings having two carbon atoms in common, such as hydrogenated products of fused polycyclic aromatic hydrocarbons.

Non-limiting examples of the bridged compounds (A3-1a) include bicyclic, tricyclic, or tetracyclic bridged hydrocarbons or bridged heterocyclic compounds, such as decahydronaphthalene, bicyclo[2.2.0]hexane, bicyclo[2.2.2]octane, bicyclo[3.2.1]octane, bicyclo[4.3.2]undecane, bicyclo[3.3.3]undecane, thujone, carane, pinane, pinene, bornane, bornylene, norbornane, norbornene, camphor, camphoric acid, camphene, tricyclene, tricyclo[5.2.1.0^{3,8}]decane, tricyclo[4.2.1.1^{2,5}]decane, exo-tricyclo[5.2.1.0^{2,6}]decane, endo-tricyclo[5.2.1.0^{2,6}]decane, tricyclo[4.3.1.1^{2,5}]undecane, tricyclo[4.2.2.1^{2,5}]undecane, endo-tricyclo[5.2.2.0^{2,6}]undecane, adamantane, 1-adamantanol, 1-chloroadamantane, 1-methyladamantane, 1,3-dimethyladamantane, 1-methoxyadamantane, 1-carboxyadamantane, 1-methoxycarbonyladamantane, 1-nitroadamantane, tetracyclo[4.4.0.1^{2,5}.1^{7,10}]dodecane, perhydroanthracene, perhydroacenaphthene, perhydrophenanthrene, perhydrophenalene, perhydroindene, and quinuclidine; and derivatives of these compounds. These bridged compounds contain a methine carbon atom at a bridgehead position, where the bridgehead position corresponds to the junction site when two rings have two atoms in common.

Non-limiting examples of the non-aromatic cyclic compounds (A3-1b) containing a ring and a hydrocarbon group bonded to the ring include alicyclic hydrocarbons containing 3 to about 15 members and having a hydrocarbon group (such as alkyl) containing 1 to about 20 (preferably 1 to 10) carbon atoms and being bonded to the ring, such as 1-methylcyclopentane, 1-methylcyclohexane, limonene, menthene, menthol, carvomenthone, and menthone; and derivatives of these compounds. The non-aromatic cyclic compounds (A3-1b) containing a ring and a hydrocarbon group bonded to the ring contain a methine carbon atom at the bonding site between the ring and the hydrocarbon group.

Non-limiting examples of the chain compounds (A3-2) containing a methine carbon atom include chain hydrocarbons containing a tertiary carbon atom, exemplified by aliphatic hydrocarbons containing 4 to about 20 (preferably, 4 to 10) carbon atoms, such as isobutane, isopentane, isohexane, 3-methylpentane, 2,3-dimethylbutane, 2-methylhexane, 3-methylhexane, 3,4-dimethylhexane, and 3-methyloctan; and derivatives of these compounds.

Non-limiting examples of the compounds (A4) containing an unsaturated bond and a carbon-hydrogen bond at a position adjacent to the unsaturated bond include (A4-1) aromatic compounds containing a methyl or methylene group at a position adjacent to the aromatic ring (at a so-called benzylic position); and (A4-2) non-aromatic compounds containing a methyl or methylene group at a position adjacent to an unsaturated bond (e.g., a carbon-carbon unsaturated bond such as a carbon-oxygen double bond).

The aromatic rings in the aromatic compounds (A4-1) may be any of aromatic hydrocarbon rings and heteroaromatic rings. Examples of the aromatic hydrocarbon rings include benzene ring; and fused carbon rings exemplified by fused carbon rings containing two to ten 4- to 7-membered carbon rings being fused to each other, such as naphthalene, azulene, indacene, anthracene, phenanthrene, triphenylene, and pyrene. Non-limiting examples of the heteroaromatic rings include oxygen-containing heterocyclic rings, sulfur-containing heterocyclic rings, and nitrogen-containing heterocyclic rings, where oxygen, sulfur, and nitrogen serve as heteroatoms. Non-limiting examples of the oxygen-containing heterocyclic rings include 5-membered rings such as furan, oxazole, and isoxazole; 6-membered rings such as 4-oxo-4H-pyran; and fused rings such as benzofuran, isobenzofuran, and 4-oxo-4H-chromene. Non-limiting examples of the sulfur-containing heterocyclic rings include 5-membered rings such as thiophene, thiazole, isothiazole, and thiadiazole; 6-membered rings such as 4-oxo-4H-thiopyran; and fused rings such as benzothiophene. Non-limiting examples of the nitrogen-containing heterocyclic rings include 5-membered rings such as pyrrole, pyrazole, imidazole, and triazole; 6-membered rings such as pyridine, pyridazine, pyrimidine, and pyrazine; and fused rings such as indole, quinoline, acridine, naphthyridine, quinazoline, and purine.

The methylene group at a position adjacent to an aromatic ring may be a methylene group constituting a non-aromatic ring bonded to the aromatic ring. In the compounds (A4-1), both a methyl group and a methylene group may be present at a position or positions adjacent to the aromatic ring.

Non-limiting examples of the aromatic compounds containing a methyl group at a position adjacent to an aromatic ring include aromatic hydrocarbons containing one to about six methyl groups substituted on an aromatic ring, such as such as toluene, o-xylene, m-xylene, p-xylene, o-t-butyltoluene, m-t-butyltoluene, p-t-butyltoluene, 1-ethyl-4-methylbenzene, 1-ethyl-3-methylbenzene, 1-isopropyl-4-methylbenzene, 1-t-butyl-4-methylbenzene, 1-methoxy-4-methylbenzene, mesitylene, pseudocumene, durene, methylnaphthalene, dimethylnaphthalene, methylanthracene, 4,4′-dimethylbiphenyl, tolualdehyde, dimethylbenzaldehyde, trimethylbenzaldehyde, toluic acid, trimethylbenzoic acid, and dimethylbenzoic acid; and heterocyclic compounds containing one to about six methyl groups substituted on a heterocyclic ring, such as 2-methylfuran, 3-methylfuran, 3-methylthiophene, 2-methylpyridine, 3-methylpyridine, 4-methylpyridine, 2,4-dimethylpyridine, 2,4,6-trimethylpyridine, 4-methylindole, 2-methylquinoline, and 3-methylquinoline.

Non-limiting examples of the aromatic compounds containing a methylene group at a position adjacent to an aromatic ring include aromatic hydrocarbons containing C₂ or higher alkyl or substituted alkyl, such as ethylbenzene, propylbenzene, butylbenzene, 1,4-diethylbenzene, and diphenylmethane; heteroaromatic compounds containing C₂ or higher alkyl or substituted alkyl, such as 2-ethylfuran, 3-propylthiophene, 4-ethylpyridine, and 4-butylquinoline; and compounds each containing an aromatic ring and a non-aromatic ring fused to an aromatic ring and containing a methylene group in the non-aromatic ring at a position adjacent to the aromatic ring, such as dihydronaphthalene, indene, indane, tetrahydronaphthalene, fluorene, acenaphthene, phenalene, indanone, and xanthene.

Non-limiting examples of the non-aromatic compounds (A4-2) containing a methyl or methylene group at a position adjacent to an unsaturated bond include (A4-2a) chain unsaturated hydrocarbons containing a methyl or methylene group at a so-called allyl position; and (A4-2b) compounds containing a methyl or methylene group at a position adjacent to carbonyl.

Non-limiting examples of the chain unsaturated hydrocarbons (A4-2a) include chain unsaturated hydrocarbons containing about 3 to about 20 carbon atoms, such as propylene, 1-butene, 2-butene, 1-pentene, 1-hexene, 2-hexene, 1,5-hexadiene, 1-octene, 3-octene, and undecatriene. Examples of the compounds (A4-2b) include ketones; and carboxylic acids and derivatives thereof. Non-limiting examples of the ketones include chain ketones such as acetone, methyl ethyl ketone, 3-pentanone, and acetophenone; and cyclic ketones such as cyclohexanone. Non-limiting examples of the carboxylic acids and derivatives thereof include acetic acid, propionic acid, butanoic acid, pentanoic acid, hexanoic acid, heptanoic acid, phenylacetic acid, malonic acid, succinic acid, and glutaric acid; and esters of them.

The alicyclic compounds (i.e., non-aromatic cyclic hydrocarbons) (A5) include (A5-1) cycloalkanes; and (A5-2) cycloalkenes.

Non-limiting examples of the cycloalkanes (A5-1) include compounds containing a 3- to 30-membered (preferably 5- to 30-membered, and particularly preferably 5- to 20-membered) cycloalkane ring, such as cyclopropane, cyclobutane, cyclopentane, cyclohexane, cycloheptane, cyclooctane, cyclononane, cyclodecane, cyclododecane, cyclotetradecane, cyclohexadecane, cyclotetracosane, and cyclotriacontane; and derivatives of them.

Examples of the cycloalkenes (A5-2) include, but are not limited to, compounds containing a 3- to 30-membered (preferably 3 to 20-membered, and particularly preferably 3 to 12-membered) cycloalkene ring, such as cyclopropene, cyclobutene, cyclopentene, cyclooctene, cyclohexene, 1-methyl-cyclohexene, isophorone, cycloheptene, and cyclododecene; cycloalkadienes such as cyclopentadiene, 1,3-cyclohexadiene, and 1,5-cyclooctadiene; cycloalkatrienes such as cyclooctatriene; and derivatives of them.

Examples of the conjugated compounds (A6) include, but are not limited to, (A6-1) conjugated dienes; (A6-2) α,β-unsaturated nitriles; α,β-unsaturated carboxylic acids and derivatives thereof (such as esters, amides; and (A6-3) acid anhydrides).

Non-limiting examples of the conjugated dienes (A6-1) include butadiene, isoprene, 2-chlorobutadiene, and 2-ethylbutadiene. Herein, the “conjugated dienes (A6-1)” also include compounds containing a double bond and a triple bond conjugated to each other, such as vinylacetylene.

Non-limiting examples of the α,β-unsaturated nitriles (A6-2) include (meth)acrylonitriles.

Non-limiting examples of the α,β-unsaturated carboxylic acids and derivatives thereof (A6-3) include (meth)acrylic acid; (meth)acrylic esters such as methyl (meth)acrylate, ethyl (meth)acrylate, isopropyl (meth)acrylate, butyl (meth)acrylate, and 2-hydroxyethyl (meth)acrylate; and (meth)acrylamide and (meth)acrylamide derivatives such as N-methylol(meth)acrylamide.

Non-limiting examples of the amine compounds (A7) include primary or secondary amines exemplified by aliphatic amines such as methylamine, ethylamine, propylamine, butylamine, dimethylamine, diethylamine, dibutylamine, ethylenediamine, 1,4-butanediamine, hydroxyamine, and ethanolamine; alicyclic amines such as cyclopentylamine and cyclohexylamine; aromatic amines such as benzyl amine and toluidine; and cyclic amines (to which an aromatic or non-aromatic ring may be fused), such as pyrrolidine, piperidine, piperazine, and indoline.

Non-limiting examples of the aromatic compounds (A8) include aromatic hydrocarbons containing at least one benzene ring, such as benzene, naphthalene, acenaphthylene, phenanthrene, anthracene, and naphthacene; and fused polycyclic aromatic hydrocarbons containing at least two (e.g., two to ten) benzene rings fused to each other. To the benzene ring or rings, any of non-aromatic carbon rings, heteroaromatic rings, and non-heteroaromatic rings may be fused. These aromatic compounds may each have one or more substituents. Specific, but non-limiting examples of such aromatic compounds having a substituent or substituents include 2-chloronaphthalene, 2-methoxynaphthalene, 1-methylnaphthalene, 2-methylnaphthalene, 1-bromoanthracene, 2-methylanthracene, 2-t-butylanthracene, 2-carboxyanthracene, 2-ethoxycarbonylanthracene, 2-cyanoanthracene, 2-nitroanthracene, and 2-methylpentalene.

Non-limiting examples of the straight chain alkanes (A9) include straight chain alkanes containing 1 to about 30 (preferably 1 to 20) carbon atoms, such as methane, ethane, propane, butane, pentane, hexane, heptane, octane, nonane, decane, dodecane, tetradecane, and hexadecane.

The olefins (A10) include α-olefins, internal olefins, and olefins containing two or more carbon-carbon double bonds, such as dienes, each of which may have one or more substituents such as hydroxy and acyloxy. Specific, but non-limiting examples of the olefins include chain olefins such as ethylene, propylene, 1-butene, 2-butene, isobutene, 1-hexene, 2-hexene, 1-acetoxy-3,7-dimethyl-2,6-octadiene, styrene, vinyltoluene, α-methylstyrene, 3-vinylpyridine, and 3-vinylthiophene; and cyclic olefins such as cyclopentene, cyclohexene, cycloheptene, cyclooctene, cyclodecene, cyclododecene, 1,4-cyclohexadiene, limonene, 1-p-menthene, 3-p-menthene, carveol, bicyclo[2.2.1]hept-2-ene, bicyclo[3.2.1]oct-2-ene, α-pinene, and 2-bornene.

Each of different substrates (A) may be used alone or in combination. Since using the oxidizer oxygen in coexistence with ozone, the present invention enables oxidation of the substrate with extremely excellent oxidizing power. The present invention thereby enables efficient oxidation even of the straight chain alkanes (A9), which are generally believed to resist oxidation, to give corresponding oxides (oxygen-containing compounds) in high yields.

In particular, the substrate (A) for use in the present invention is preferably selected from hydrocarbons. Non-limiting examples of the hydrocarbons include aliphatic hydrocarbons, alicyclic hydrocarbons, and aromatic hydrocarbons.

Non-limiting examples of the aliphatic hydrocarbons include straight chain alkanes such as ethane, propane, butane, pentane, hexane, heptane, octane, nonane, decane, undecane, dodecane, tridecane, tetradecane, pentadecane, hexadecane, heptadecane, octadecane, nonadecane, icosane, henicosane, docosane, triacontane, and tetracontane, of which C₃-C₃₀, preferably C₄-C₂₀, straight chain alkanes are typified; branched chain alkanes such as 2-methylpropane, 2-methylbutane, 2,2-dimethylpropane, 2-methylpentane, 3-methylpentane, 2,3-dimethylbutane, 2-methylhexane, 3-methylhexane, 3,4-dimethylhexane, and 3-methyloctane, of which C₃-C₃₀, preferably C₄-C₂₀, branched chain alkanes are typified; and straight or branched chain alkenes and alkadienes such as propylene, isobutylene, 1-pentene, 1-hexene, 2-hexene, 1-heptene, 1-octene, 1-decene, 1-dodecene, 1-tetradecene, and 1,4-hexadiene, of which C₃-C₃₀, preferably C₄-C₂₀, straight or branched chain alkenes and alkadienes are typified.

Non-limiting examples of the alicyclic hydrocarbons include cycloalkanes such as cyclopropane, cyclobutane, cyclopentane, cyclohexane, cycloheptane, cyclooctane, cyclononane, cyclodecane, cyclododecane, cyclotetradecane, cyclohexadecane, and cyclotriacontane, of which 3- to 30-membered (preferably 5- to 30-membered, and particularly preferably 5- to 20-membered) cycloalkanes are typified; cycloalkenes such as cyclopropene, cyclobutene, cyclopentene, cyclooctene, cyclohexene, cycloheptene, and cyclododecene, of which 3- to 30-membered (preferably 3- to 20-membered, and particularly preferably 3- to 12-membered) cycloalkenes are typified; cycloalkadienes such as cyclopentadiene, 1,3-cyclohexadiene, and 1,5-cyclooctadiene; cycloalkatrienes such as cyclooctatriene; and bicyclic, tricyclic, or tetracyclic bridged hydrocarbons such as decalin (decahydronaphthalene), bicyclo[2.2.0]hexane, bicyclo[2.2.2]octane, bicyclo[3.2.1]octane, bicyclo[4.3.2]undecane, bicyclo[3.3.3]undecane, norbornane, norbornene, tricyclo[5.2.1.0^{2,6}]decane, tricyclo[6.2.1.0^{2,7}]undecane, adamantane, perhydroanthracene, perhydroacenaphthene, perhydrophenanthrene, perhydrophenalene, and perhydroindene.

Non-limiting examples of the aromatic hydrocarbons include C₆-C₂₀ aromatic hydrocarbons such as benzene, naphthalene, and anthracenes.

The hydrocarbons may each have, as substituents, one or more groups selected from the group consisting of aliphatic hydrocarbon groups, alicyclic hydrocarbon groups, and aromatic hydrocarbon groups.

Non-limiting examples of the aliphatic hydrocarbon groups include monovalent or multivalent aliphatic hydrocarbon groups which are groups resulting from removing one or more hydrogen atoms each from the structural formulae of the aliphatic hydrocarbons. Non-limiting examples of the monovalent aliphatic hydrocarbon groups include C₁-C₁₀ straight or branched chain alkyls such as methyl, ethyl, propyl, isopropyl, butyl, isobutyl, s-butyl, t-butyl, pentyl, hexyl, octyl, and decyl; C₂-C₁₀ alkenyls such as vinyl, isopropenyl, and 1-butenyl; and C₂-C₁₀ alkynyls such as ethynyl and propynyl. Non-limiting examples of divalent aliphatic hydrocarbon groups include C₁-C₁₀ straight or branched chain alkylene groups such as methylene, ethylene, propylene, trimethylene, isopropylidene, and tetramethylene groups. Non-limiting examples of the trivalent aliphatic hydrocarbon groups include C₁-C₁₀ alkanetriyls such as 1,2,3-propanetriyl.

Examples of the alicyclic hydrocarbon groups include monovalent or multivalent alicyclic hydrocarbon groups which are groups resulting from removing one or more hydrogen atoms each from the structural formulae of the alicyclic hydrocarbons. Non-limiting examples of the alicyclic hydrocarbon groups include cycloalkyls such as cyclopentyl and cyclohexyl; cycloalkenyls such as cyclopentenyl and cyclohexenyl; and bridged hydrocarbon groups such as adamant-1-yl, norborn-2-yl, and norbornane-7,7-diyl.

Examples of the aromatic hydrocarbon groups include monovalent or multivalent aromatic hydrocarbon groups which are groups resulting from removing one or more hydrogen atoms each from the structural formulae of the aromatic hydrocarbons. Non-limiting examples of the aromatic hydrocarbon groups include phenyl, 1-naphthyl, 1,2-phenylene, 1,3-phenylene, and 1,4-phenylene groups.

The “hydrocarbons” also include compounds each including an alicyclic hydrocarbon and an aromatic hydrocarbon fused to each other in such a manner as to have two carbon atoms in common. Non-limiting examples of compounds of this category include indane, tetrahydronaphthalene, fluorene, and acenaphthene.

The hydrocarbons may each have one or more substituents other than hydrocarbon groups within ranges not adversely affecting the reaction, or not.

The hydrocarbons for use as the reaction substrate in the present invention may contain carbon atoms in any number not limited, but contain preferably about 2 to about 30, more preferably 3 to 25, and furthermore preferably 4 to 20 carbon atoms.

Preferred, but non-limiting examples of the hydrocarbons for use in the present invention include straight chain alkanes such as propane, butane, pentane, hexane, heptane, octane, nonane, decane, undecane, dodecane, tridecane, tetradecane, pentadecane, hexadecane, heptadecane, octadecane, nonadecane, and icosane, of which C₃-C₃₀, preferably C₄-C₂₀, straight chain alkanes are typified; branched chain alkanes such as 2-methylpropane, 2-methylbutane, 2,2-dimethylpropane, 2-methylpentane, 3-methylpentane, 2,3-dimethylbutane, 2-methylhexane, 3-methylhexane, 3,4-dimethylhexane, and 3-methyloctane, of which C₃-C₃₀, preferably C₄-C₂₀, branched chain alkanes are typified; cycloalkanes such as cyclopentane and cyclohexane, of which 3- to 30-membered (preferably 5- to 30-membered, and particularly preferably 5- to 20-membered) cycloalkanes are typified; aromatic hydrocarbons having one or more alkyls (such as C₁-C₆ alkyls) bonded to an aromatic ring, such as toluene, o-xylene, m-xylene, p-xylene, o-t-butyltoluene, m-t-butyltoluene, p-t-butyltoluene, 1-ethyl-4-methylbenzene, 1-ethyl-3-methylbenzene, 1-isopropyl-4-methylbenzene, 1-t-butyl-4-methylbenzene, mesitylene, pseudocumene, durene, methylnaphthalene, dimethylnaphthalene, methylanthracene, 4,4′-dimethylbiphenyl, ethylbenzene, propylbenzene, butylbenzene, and 1,4-diethylbenzene; and compounds each containing an alicyclic hydrocarbon and an aromatic hydrocarbon fused to each other in such a manner as to have two carbon atoms in common, such as tetrahydronaphthalene and fluorene.

In particular, the present invention is especially useful in oxidation using straight chain alkanes as the substrate, because the present invention enables oxidation of straight chain alkanes in high yields, where the straight chain alkanes are hardly oxidized in high yields by conventional methods.

Oxidizer

The present invention uses the oxidizer oxygen in the coexistence with ozone. With the present invention, the reaction is performed in the presence of oxygen and ozone or in a stream of oxygen gas and ozone gas. The use of the oxidizer oxygen in combination with ozone can promote hydrogen withdrawal from the substrate (A) to activate a radical reaction. This can promote the oxidation reaction even under mild conditions. Under such mild conditions, the reaction pressure is typically at normal atmospheric pressure, and the reaction temperature is typically from around room temperature to around 200° C., preferably 50° C. to 130° C., and particularly preferably 60° C. to 100° C. When the reaction is performed in a stream of oxygen gas and ozone gas, the ozone-containing oxygen gas may contain the ozone gas in a proportion of typically about 0.1 to about 10 volume percent relative to the oxygen gas, from the viewpoints of reactivity and economic efficiency.

The oxygen for use herein is preferably molecular oxygen. The molecular oxygen may be selected from pure oxygen; oxygen diluted with an inert gas such as nitrogen, helium, argon, or carbon dioxide gas; and air at normal atmospheric pressure or under pressure (at 1 to 100 atmospheric pressures). The molecular oxygen may be used in an amount not limited, as long as being 1 mole or more per mole of the substrate (A) used as the substrate.

Ozone in the present invention acts as a radical generator (radical precursor). The ozone for use herein is preferably ozone gas. The ozone gas may be used in an amount not limited, as long as being 0.01 mole or more per mole of the substrate (A). The ozone gas may be fed discontinuously or continuously, as long as the reaction proceeds smoothly. In combination with ozone, another radical generator such as azobisisobutyronitrile (AIBN) may be used.

Solid Acid Catalyst

The solid acid catalyst according to the present invention for oxygen-ozone-coexisting oxidation is a catalyst including a transition metal in the form of an elementary substance, a compound, or an ion, and a support supporting the transition metal, where the support includes, at least in its surface, a strong acid or super strong acid having a Hammett acidity function (H₀) of −9 or less. The solid acid catalyst according to the present invention for oxygen-ozone-coexisting oxidation may also be a catalyst including a transition metal in the form of an elementary substance, a compound, or an ion, and a support supporting the transition metal, where the support has, at least in its surface, at least one strong acid or super strong acid selected from the group consisting of sulfuric acid, sulfated metal oxides, noble metals/sulfated metal oxides, metal oxide super strong acids, and fluorinated sulfonic acid resins.

Non-limiting examples of the strong acid or super strong acid having a Hammett acidity function (H₀) of −9 or less include concentrated sulfuric acid, solid strong acids, and solid super strong acids (acids having a Hammett acidity function (H₀) of less than −11.93). The concentrated sulfuric acid has a Hammett acidity function H₀ of −9.88 at 96%, of −10.27 at 98%, and of −11.93 at 100%.

Non-limiting examples of the solid strong acids or solid super strong acids include immobilized liquid super strong acids each including a liquid super strong acid (such as SbF₅, BF₃, BF—SbF₅, FSO₃H—SbF₅, or TaF₅) supported on a solid (such as Al₂O₃, SiO₂, zeolite, SiO₂—Al₂O₃, a polymer, graphite, or a metal); binary metal salts each prepared by grinding and mixing AlCl₃ or AlBr₃ with one of CuSO₄, CuCl₂, Ti₂ (SO₄)₃, and TiCl₃; sulfated metal oxides each formed by allowing a metal oxide (such as Fe₂O₃, TiO₂, ZrO₂, HfO₂, SnO₂, Al₂O₃, or SiO₂) to adsorb sulfate ions and firing the resulting article to allow the sulfate ions to be supported by and bonded to the metal oxide; noble metals/sulfated metal oxides as the sulfated metal oxides mixed with a noble metal such as Ir or Pt; metal oxide super strong acids formed by allowing a metal oxide (such as ZrO₂, SnO₂, TiO₂, or Fe₂O₃) to adsorb, for example, WO₃, MoO₃, or B₂O₃; strongly acidic or super acidic ion exchange resins such as non-porous or porous ion exchange resins each containing a strong acid group or super strong acid group such as —CF₃, CF₂, or SO₃H; heteropolyacids such as polyacids containing an element such as P, Mo, V, W, or Si. Non-limiting examples of the strongly acidic or super acidic ion exchange resins include fluorinated sulfonic acid resins (fluorocarbon resins containing, in a side chain, a group containing a sulfonic group (sulfo-containing group)). Preferred, but non-limiting examples of the fluorinated sulfonic acid resins include copolymers between a sulfo-containing perfluorovinyl ether monomer and tetrafluoroethylene, such as Nafion® NR50 (supplied by Aldrich) and Nafion® H (supplied by E. I. du Pont de Nemours & Co.).

In particular, the support for use in the present invention is preferably selected from sulfated metal oxides, noble metals/sulfated metal oxides, metal oxide super strong acids, strongly acidic or super acidic ion exchange resins (such as fluorinated sulfonic acid resins), and especially preferably selected from sulfated metal oxides, noble metals/sulfated metal oxides, and fluorinated sulfonic acid resins. The sulfated metal oxides, noble metals/sulfated metal oxides, and fluorinated sulfonic acid resins have particularly high acid strengths and can support the transition metal compound more effectively. Of the sulfated metal oxides, preferred examples include sulfated zirconia, sulfated tin, and sulfated hafnium. Of the noble metals/sulfated metal oxides, preferred examples include Pt/sulfated zirconia, Ir/sulfated zirconia, and Pd/sulfated zirconia. Among them, the support for use herein is advantageously selected from sulfated zirconia and fluorinated sulfonic acid resins, each of which is industrially easily available. In particular, the support is preferably selected from fluorinated sulfonic acid resins such as Nafion® and other copolymers between a sulfo-containing perfluorovinyl ether monomer and tetrafluoroethylene.

Examples of the “support including or bearing, at least in its surface, a strong acid or super strong acid having a Hammett acidity function (H₀) of −9 or less” include, but are not limited to, (i) strong acids or super strong acids having a Hammett acidity function (H₀) of −9 or less used as supports by themselves; and (ii) supports each including a solid and a layer disposed on the solid, where the layer (coating) is of any of the strong acids or super strong acids having a Hammett acidity function (H₀) of −9 or less.

Non-limiting examples of the solid in the supports (ii) include inorganic substances exemplified by inorganic oxides (including complex oxides) such as Al₂O₃, SiO₂, zeolite, SiO₂—Al₂O₃, and glass, activated carbon, graphite, and metals; and resins (polymers). Non-limiting examples of the resins include fluorocarbon resins such as Teflon® and Daiflon, polyester resins, polyolefins resins, polycarbonate resins, polystyrene resins, polyamide resins, and polyimide resins.

The support in the present invention is not limited in shape and particle size and can be selected from supports of pellet-like, powdery, granular, film-like, tubular, and any other shapes generally employed in solid catalysts. The support may be present as a layer (coating) on the inner wall of a reactor such as a flow reactor.

In particular, the support is preferably selected from supports each including a fluorinated sulfonic acid resin and being in the form of a pellet or a particle; and supports each including a solid, and a layer of a fluorinated sulfonic acid resin disposed on the solid. Non-limiting examples of the solid include resins; and inorganic materials such as inorganic oxides.

The transition metal is often selected from metal elements belonging to Groups 3 to 12 of the periodic table.

Non-limiting examples of the metal elements include, of the periodic table, Group 3 elements such as Sc, lanthanoids, and actinoids; Group 4 elements such as Ti, Zr, and Hf; Group 5 elements such as V; Group 6 elements such as Cr, Mo, and W; Group 7 elements such as Mn; Group 8 elements such as Fe and Ru; Group 9 elements such as Co and Rh; Group 10 elements such as Ni, Pd, and Pt; Group 11 elements such as Cu; and Group 12 elements such as Zn. Preferred, but non-limiting examples of the metal elements include elements belonging to Groups 5 to 11 of the periodic table, of which elements belonging to Groups 5 to 9, such as Co, Mn, Fe, V, and Mo are typified. Among them, Co, Mn, Fe, Zr, Ce, V, Mo, and W are preferred, of which Co, Mn, Fe, Zr, and V are particularly preferred. The metal elements may each have any valence not limited and have a valence of typically 0 to about 6, preferably 2 to 5, and more preferably 2 or 3.

Examples of the transition metal compound include, of the metal elements, inorganic compounds and organic compounds. Non-limiting examples of the inorganic compounds include elementary substances, hydroxides, oxides (including complex oxides), halides (fluorides, chlorides, bromides, and iodides), oxoacid salts (such as nitrates, sulfates, phosphates, borates, and carbonates), isopolyacid salts, and heteropolyacid salts. Non-limiting examples of the organic compounds include complexes, and organic acid salts exemplified by acetates, propionates, cyanides, naphthenates, and stearates; alkylsulfonates such as methanesulfonates, ethanesulfonates, octanesulfonates, and dodecanesulfonates, of which C₆-C₁₈ alkylsulfonates are typified; and optionally alkyl-substituted arylsulfonates such as benzenesulfonates, p-toluenesulfonates, naphthalenesulfonates, decylbenzenesulfonates, and dodecylbenzenesulfonates, of which C₆-C₁₈ alkyl-arylsulfonates are typified. Non-limiting examples of ligands constituting the complexes include OH (hydroxo), alkoxys (such as methoxy, ethoxy, propoxy, and butoxy), acyls (such as acetyl and propionyl), alkoxycarbonyls (such as methoxycarbonyl and ethoxycarbonyl), acetylacetonato, cyclopentadienyl, halogen atoms (such as chlorine and bromine), CO, CN, oxygen atom, H₂O (aquo), phosphorus compounds exemplified by phosphines (such as triphenylphosphine and other triarylphosphines), and nitrogen-containing compounds such as NH₃ (ammine), NO, NO₂ (nitro), NO₃ (nitrato), ethylenediamine, diethylenetriamine, pyridine, and phenanthroline.

Specific, but non-limiting examples of the transition metal compound include, when taking cobalt compounds as an example, divalent or trivalent cobalt compounds exemplified by inorganic compounds such as cobalt hydroxides, cobalt oxides, cobalt chlorides, cobalt bromides, cobalt nitrates, cobalt sulfates, and cobalt phosphate; organic acid salts such as cobalt acetate, cobalt naphthenate, and cobalt stearate; and complexes such as acetylacetonatocobalt. Non-limiting examples of manganese compounds include divalent to pentavalent manganese compounds exemplified by inorganic compounds such as manganese hydroxides, manganese oxides, manganese chlorides, and manganese sulfates; and complexes such as acetylacetonatomanganese. Examples of compounds of other transition metal elements include compounds corresponding to the cobalt or manganese compounds. The catalyst may include each of different transition metal compounds alone or in combination. In an embodiment, the support preferably includes two or more transition metal compounds having different valences (e.g., a divalent metal compound and a trivalent metal compound) in combination. The transition metal compound for use in the present invention preferably includes one or more compounds selected from compounds each containing, as the transition metal, one selected from Co (cobalt), Mn (manganese), Fe (iron), Zr (zirconium), Ce (cerium), V (vanadium), Mo (molybdenum), and W (tungsten), and particularly one selected from Co, Mn, Fe, Zr, and V. In particular, the transition metal compound preferably includes one or more compounds selected from cobalt compounds and manganese compounds (of which organic acid salts are typified) and especially preferably includes both a cobalt compound and a manganese compound in combination. This is preferred for restraining deterioration in catalytic activity.

The transition metal ion for use herein preferably includes one or more ions selected from cobalt ions, manganese ions, iron ions, zirconium ions, and cerium ions and particularly preferably includes a cobalt ion, or a manganese ion, or both (a cobalt ion in combination with a manganese ion).

The supporting of the transition metal in the form of an elementary substance, a compound, or an ion onto the support can be performed by any of common processes such as impregnation, firing, precipitation, and ion exchange processes. Assume that the support is disposed as a layer on the inner wall of a reactor such as a flow reactor. In this case, the supporting of the transition metal onto the support can be performed by allowing a solution of a transition metal compound to flow through the reactor.

The transition metal (in the form of an elementary substance, a compound, or an ion) may be supported in an amount of typically about 0.001 to about 20 weight percent, preferably about 0.01 to about 20 weight percent, and particularly about 0.1 to about 10 weight percent in terms of metal atoms typically in a transition metal compound, relative to the support. The support, when ion-exchanged, may be ion-exchanged with a transition metal ion in an amount of typically about 0.001 to about 20 weight percent, preferably about 0.01 to about 20 weight percent, and particularly about 0.1 to about 10 weight percent in terms of metal atoms in the transition metal ion, relative to the support.

Method for Producing Oxide

The method according to the present invention for producing an oxide includes oxidizing the substrate (A) in the coexistence of oxygen and ozone in the presence of the solid acid catalyst.

The present invention may employ a solvent, or not. The solvent may be used in such an amount that the total amount of the substrate (A) and an oxide or oxides of the substrate (A) is typically about 70 weight percent or more, preferably 85 weight percent or more, and particularly preferably 95 weight percent or more, based on the total amount of liquid components in the reaction system. In an embodiment, approximately no solvent is used. This configuration eliminates or minimizes the need for separating the reaction product oxide or oxides from the solvent and contributes to simplification of the production process.

Non-limiting examples of the solvent include organic acids such as acetic acid and propionic acid; nitriles such as acetonitrile, propionitrile, and benzonitrile; amides such as formamide, acetamide, dimethylformamide, and dimethylacetamide; aliphatic hydrocarbons such as hexane and octane; halogenated hydrocarbons such as chloroform, dichloromethane, dichloroethane, carbon tetrachloride, chlorobenzene, and trifluoromethylbenzene; nitro compounds such as nitrobenzene, nitromethane, and nitroethane; esters such as ethyl acetate and butyl acetate; and mixtures of these solvents.

When the reaction is performed according to a batch system, the solid acid catalyst may be used in an amount of typically about 0.00001 to about 10 mole percent, and preferably 0.2 to 2 mole percent, in terms of the transition metal supported on the support in the solid acid catalyst, relative to the substrate (A). When two or more different solid acid catalysts are used, the term “amount” refers to the total amount of them.

Imide Compound Having Cyclic Imide Skeleton

The present invention may employ an imide compound having a cyclic imide skeleton in combination with the solid acid catalyst, so as to further promote the oxidation reaction of the substrate (A). The imide compound having a cyclic imide skeleton is hereinafter also simply referred to as an “imide compound”. Non-limiting examples of the imide compound having a cyclic imide skeleton include compounds represented by Formula (I):

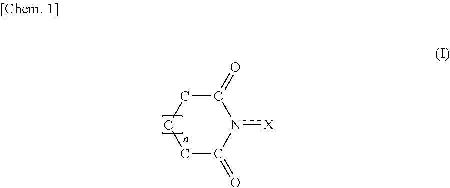

In Formula (I), n represents one of 0 and 1. X is selected from oxygen and an —OR group, where R is selected from hydrogen and a hydroxy-protecting group. The bond between the nitrogen atom and X is selected from a single bond and a double bond. The imide compounds may each have two or more cyclic imide skeletons represented by Formula (I). In the imide compounds, when X is an —OR group, and R is a hydroxy-protecting group, two or more cyclic N-oxyimide skeletons may be bonded through R. The term “cyclic N-oxyimide skeleton” refers to a moiety of the cyclic imide skeleton excluding R.

Non-limiting examples of the hydroxy-protecting group represented by R in Formula (I) include alkyls (exemplified by C₁-C₄ alkyls such as methyl and t-butyl), alkenyls (such as allyl), cycloalkyls (such as cyclohexyl), aryls (such as 2,4-dinitrophenyl), aralkyls (such as benzyl, 2,6-dichlorobenzyl, 3-bromobenzyl, 2-nitrobenzyl, and triphenylmethyl); groups capable of forming an acetal or hemiacetal group with hydroxy; acyls; and other hydroxy-protecting groups. Non-limiting examples of the groups capable of forming an acetal or hemiacetal group with hydroxy include substituted methyls such as methoxymethyl, methylthiomethyl, benzyloxymethyl, t-butoxymethyl, 2-methoxyethoxymethyl, 2,2,2-trichloroethoxymethyl, bis(2-chloroethoxy)methyl, and 2-(trimethylsilyl)ethoxymethyl; substituted ethyls such as 1-ethoxyethyl, 1-methyl-1-methoxyethyl, 1-isopropoxyethyl, 2,2,2-trichloroethyl, and 2-methoxyethyl; tetrahydropyranyls; tetrahydrofuranyls; and 1-hydroxyalkyls such as 1-hydroxyethyl, 1-hydroxyhexyl, 1-hydroxydecyl, 1-hydroxyhexadecyl, and 1-hydroxy-1-phenylmethyl. Non-limiting examples of the acyls include aliphatic saturated or unsaturated acyls exemplified by C₁-C₂₀ aliphatic acyls such as formyl, acetyl, propionyl, butyryl, isobutyryl, valeryl, pivaloyl, hexanoyl, heptanoyl, octanoyl, nonanoyl, decanoyl, lauroyl, myristoyl, palmitoyl, and stearoyl; acetoacetyl; alicyclic acyls exemplified by cycloalkanecarbonyls such as cyclopentanecarbonyl and cyclohexanecarbonyl; and aromatic acyls such as benzoyl and naphthoyl. Non-limiting examples of the other hydroxy-protecting groups include sulfonyls such as methanesulfonyl, ethanesulfonyl, trifluoromethanesulfonyl, benzenesulfonyl, p-toluenesulfonyl, and naphthalenesulfonyl; alkoxycarbonyls exemplified by C₁-C₄ alkoxy-carbonyls such as methoxycarbonyl, ethoxycarbonyl, and t-butoxycarbonyl; aralkyloxycarbonyls such as benzyloxycarbonyl and p-methoxybenzyloxycarbonyl; substituted or unsubstituted carbamoyls such as carbamoyl, methylcarbamoyl, and phenylcarbamoyl; groups resulting from removing an OH group from inorganic acids (such as sulfuric acid, nitric acid, phosphoric acid, and boric acid); dialkylphosphinothioyls such as dimethylphosphinothioyl; diarylphosphinothioyls such as diphenylphosphinothioyl; and substituted silyls such as trimethylsilyl, t-butyldimethylsilyl, tribenzylsilyl, and triphenylsilyl.

Assume that X is an —OR group, and two or more cyclic N-oxyimide skeletons (each a moiety of the cyclic imide skeleton excluding R) are bonded through R. In this case, non-limiting examples of R include polycarboxylic acid acyls such as oxalyl, malonyl, succinyl, glutaryl, adipoyl, phthaloyl, isophthaloyl, and terephthaloyl; carbonyl; and multivalent hydrocarbon groups such as methylene, ethylidene, isopropylidene, cyclopentylidene, cyclohexylidene, and benzylidene groups, of which groups capable of forming an acetal bond with two hydroxy groups are typified.

Preferred, but non-limiting examples of R include hydrogen; groups capable of forming an acetal or hemiacetal group with hydroxy; acyls, sulfonyls, alkoxycarbonyls, carbamoyls, and other groups resulting from removing an OH group form acids such as carboxylic acids, sulfonic acids, carbonic acid, carbamic acid, sulfuric acid, phosphoric acid, and boric acid, and other hydrolyzable protecting groups capable of leaving by hydrolysis.

In Formula (I), n is selected from 0 and 1. Namely, Formula (I) represents a 5-membered cyclic imide skeleton when n is 0, and represents a 6-membered cyclic imide skeleton when n is 1.

Representative, but non-limiting examples of the imide compounds include compounds represented by Formula (1):

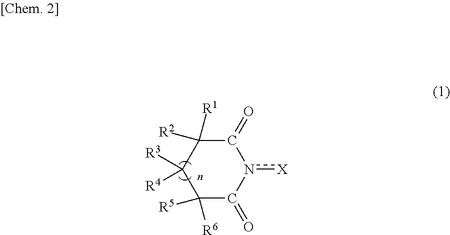

In Formula (1), n is selected from 0 and 1; X is selected from oxygen and an —OR group where R is selected from hydrogen and a hydroxy-protecting group; and R¹, R², R³, R⁴, R⁵, and R⁶ are each, identically or differently, selected from hydrogen, halogen, alkyl, aryl, cycloalkyl, hydroxy, alkoxy, carboxy, substituted oxycarbonyl, acyl, and acyloxy. At least two of R¹, R², R³, R⁴, R⁵, and R⁶ may be linked to each other to form a double bond and/or to form a ring with a carbon atom or atoms constituting the cyclic imide skeleton. On the substituent R¹, R², R³, R⁴, R⁵, or R⁶, and/or on the double bond formed by at least two of R¹, R², R³, R⁴, R⁵, and R⁶ linked to each other, and/or on the ring formed by at least two of R¹, R², R³, R⁴, R⁵, and R⁶ with a carbon atom or atoms constituting the cyclic imide skeleton, one or more occurrences of the cyclic imido group specified in Formula (1) may be further formed.

In the imide compounds represented by Formula (1), non-limiting examples of the halogen as the substituents R¹, R², R³, R⁴, R⁵, and R⁶ include iodine, bromine, chlorine, and fluorine. Non-limiting examples of the alkyl include linear or branched chain alkyls containing 1 to about 30 carbon atoms, such as methyl, ethyl, propyl, isopropyl, butyl, isobutyl, s-butyl, t-butyl, hexyl, decyl, dodecyl, tetradecyl, and hexadecyl, of which alkyls containing 1 to about 20 carbon atoms are typified.

Non-limiting examples of the aryl include phenyl and naphthyl, and non-limiting examples of the cycloalkyl include cyclopentyl and cyclohexyl. Non-limiting examples of the alkoxy include alkoxys containing 1 to about 30 carbon atoms, such as methoxy, ethoxy, isopropoxy, butoxy, t-butoxy, hexyloxy, octyloxy, decyloxy, dodecyloxy, tetradecyloxy, and octadecyloxy, of which alkoxys containing 1 to about 20 carbon atoms are typified.

Non-limiting examples of the substituted oxycarbonyl include C₁-C₃₀ alkoxy-carbonyls such as methoxycarbonyl, ethoxycarbonyl, isopropoxycarbonyl, butoxycarbonyl, t-butoxycarbonyl, hexyloxycarbonyl, decyloxycarbonyl, and hexadecyloxycarbonyl, of which C₁-C₂₀ alkoxy-carbonyls are typified; cycloalkyloxycarbonyls such as cyclopentyloxycarbonyl and cyclohexyloxycarbonyl, of which 3 to 20-membered cycloalkyloxy-carbonyls are typified; aryloxycarbonyls such as phenyloxycarbonyl and naphthyloxycarbonyl, of which C₆-C₂₀ aryloxy-carbonyls are typified; and aralkyloxycarbonyls such as benzyloxycarbonyl, of which C₇-C₂₁ aralkyloxy-carbonyls are typified.

Non-limiting examples of the acyl include aliphatic saturated or unsaturated acyls exemplified by C₁-C₃₀ aliphatic acyls such as formyl, acetyl, propionyl, butyryl, isobutyryl, valeryl, pivaloyl, hexanoyl, octanoyl, decanoyl, lauroyl, myristoyl, palmitoyl, and stearoyl, of which C₁-C₂₀ aliphatic acyls are typified; acetoacetyl; alicyclic acyls exemplified by cycloalkanecarbonyls such as cyclopentanecarbonyl and cyclohexanecarbonyl; and aromatic acyls such as benzoyl and naphthoyl.

Non-limiting examples of the acyloxy include aliphatic saturated or unsaturated acyloxys exemplified by C₁-C₃₀ aliphatic acyloxys such as formyloxy, acetyloxy, propionyloxy, butyryloxy, isobutyryloxy, valeryloxy, pivaloyloxy, hexanoyloxy, octanoyloxy, decanoyloxy, lauroyloxy, myristoyloxy, palmitoyloxy, and stearoyloxy, of which C₁-C₂₀ aliphatic acyloxys are typified; acetoacetyloxy; alicyclic acyloxys exemplified by cycloalkanecarbonyloxys such as cyclopentanecarbonyloxy and cyclohexanecarbonyloxy; and aromatic acyloxys such as benzoyloxy and naphthoyloxy.

At least two of the substituents R¹, R², R³, R⁴, R⁵, and R⁶ may be linked to each other to form a ring with a carbon atom or atoms constituting the cyclic imide skeleton. Non-limiting examples of the ring include 5- to 12-membered rings, of which 6- to 10-membered rings are particularly preferred. The rings include hydrocarbon rings, heterocyclic rings, and fused heterocyclic rings. Specific, but non-limiting examples of such rings include non-aromatic alicyclic rings exemplified by optionally substituted cycloalkane rings such as cyclohexane ring, and optionally substituted cycloalkene rings such as cyclohexene ring; non-aromatic bridged rings exemplified by optionally substituted bridged hydrocarbon rings such as 5-norbornene ring; and optionally substituted aromatic rings (including fused rings) such as benzene and naphthalene rings. Non-limiting examples of the substituents which the rings may have include alkyls, haloalkyls, hydroxys, alkoxys, carboxys, substituted oxycarbonyls, acyls, acyloxys, nitro, cyano, aminos, and halogens.

On the substituent R¹, R², R³, R⁴, R⁵, or R⁶, and/or on the double bond formed by at least two of R¹, R², R³, R⁴, R⁵, and R⁶ linked to each other, and/or on the ring formed by at least two of R¹, R², R³, R⁴, R⁵, and R⁶ with a carbon atom or atoms constituting the cyclic imide skeleton, one or more occurrences of the cyclic imido group specified in Formula (1) may be further formed. For example, when R¹, R², R³, R⁴, R⁵, or R⁶ is C₂ or higher alkyl, the cyclic imido group may be formed as including adjacent two carbon atoms constituting the alkyl. When at least two of R¹, R², R³, R⁴, R⁵, and R⁶ are linked to each other to form a double bond, the cyclic imido group may be formed as including the double bond. When at least two of R¹, R², R³, R⁴, R⁵, and R⁶ are linked to each other to form a ring with a carbon atom or atoms constituting the cyclic imide skeleton, the cyclic imido group may be formed as including adjacent two carbon atoms constituting the ring.

Preferred, but non-limiting examples of the imide compounds include compounds represented by following formulae. In the formulae, R¹¹ to R¹⁶ are each, identically or differently, selected from hydrogen, halogen, alkyl, aryl, cycloalkyl, hydroxy, alkoxy, carboxy, substituted oxycarbonyl, acyl, and acyloxy; R¹⁷ to R²⁶ are each, identically or differently, selected from hydrogen, alkyl, haloalkyl, hydroxy, alkoxy, carboxy, substituted oxycarbonyl, acyl, acyloxy, nitro, cyano, amino, and halogen, where adjacent two of R¹⁷ to R²⁶ may be bonded to each other to form the 5-membered or 6-membered cyclic imide skeleton specified in Formula (1c), (1d), (1e), (1f), (1h), or (1i); A is selected from a methylene group and oxygen; and X is as defined above.

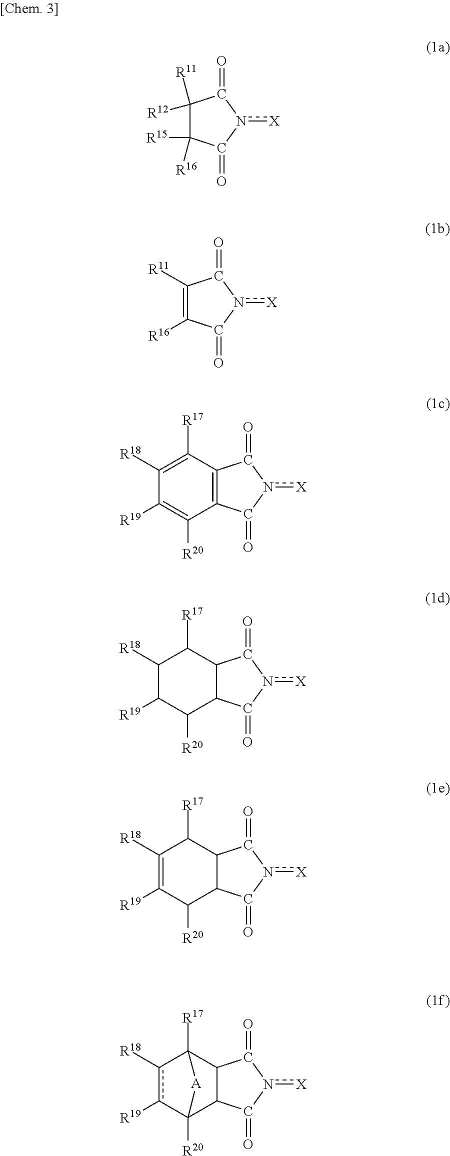

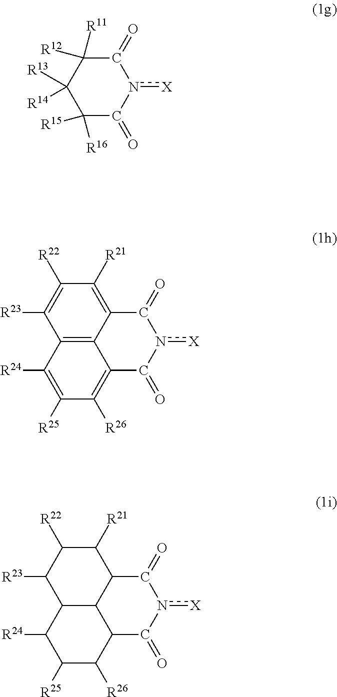

Non-limiting examples of the halogen, alkyl, aryl, cycloalkyl, hydroxy, alkoxy, carboxy, substituted oxycarbonyl, acyl, and acyloxy as the substituents R¹¹ to R¹⁶ are as with the corresponding groups in R¹¹ to R¹⁶.

As the substituents R¹⁷ to R²⁶, non-limiting examples of the alkyl are alkyls as with the above-exemplified alkyls, of which alkyls containing 1 to about 6 carbon atoms are preferred, and lower alkyls containing 1 to 4 carbon atoms are particularly preferred; non-limiting examples of the haloalkyl include haloalkyls containing 1 to about 4 carbon atoms, such as trifluoromethyl; non-limiting examples of the alkoxy include alkoxys as above, of which lower alkoxys containing 1 to about 4 carbon atoms are typified; and non-limiting examples of the substituted oxycarbonyl include substituted oxycarbonyls as above, such as alkoxycarbonyls, cycloalkyloxycarbonyls, aryloxycarbonyls, and aralkyloxycarbonyls. Non-limiting examples of the acyl include acyls as above, such as aliphatic saturated or unsaturated acyls, acetoacetyl, alicyclic acyls, and aromatic acyls; and non-limiting examples of the acyloxy include acyloxys as above, such as aliphatic saturated or unsaturated acyloxys, acetoacetyloxy, alicyclic acyloxys, and aromatic acyloxys. Non-limiting examples of the halogen include fluorine, chlorine, and bromine. In particular, the substituents R¹⁷ to R²⁶ are preferably selected from hydrogen, lower alkyls containing 1 to about 4 carbon atoms, carboxys, substituted oxycarbonyls, nitros, and halogens.

The present invention can oxidize the substrate (A) with extremely excellent oxidizing power because of using the oxidizer oxygen in the coexistence with ozone. The present invention can therefore allow the reaction to proceed rapidly under solvent-free conditions to give an oxide or oxides efficiently even when the imide compound has a solubility parameter (SP) of typically greater than 26 (MPa)^{1/2} (preferably from greater than 26 (MPa)^{1/2} to 40 (MPa)^{1/2}). The solubility parameter herein is a value determined by the Fedors method at such a temperature (25° C.) that the oxygen atom (—O—) constituting an ester bond has an energy of vaporization of 3350 J/mol and a molar volume of 3.8 cm³/mol. The solubility parameter SP can be determined by methods described in literature (see R. F. Fedors, Polym. Eng. Sci., 14(2), 147(1974); E. A. Grulke, Polymer Handbook, VII/675; and Yuji HARAZAKI, Coating Technology, 3, 129(1987)).

Of the preferred imide compounds, representative, but non-limiting examples of compounds having a 5-membered cyclic imide skeleton include compounds of Formula (1) in which X is an —OR group, and R is hydrogen, such as N-hydroxysuccinimide, N-hydroxy-α-methylsuccinimide, N-hydroxy-α,α-dimethylsuccinimide, N-hydroxy-α,β-dimethylsuccinimide, N-hydroxy-α,α,β,β-tetramethylsuccinimide, N-hydroxymaleimide, N-hydroxyhexahydrophthalimide, N,N′-dihydroxycyclohexanetetracarboxylic diimide, N-hydroxyphthalimide, N-hydroxytetrabromophthalimide, N-hydroxytetrachlorophthalimide, HET acid N-hydroxyimide (N-hydroxy-1,4,5,6,7,7-hexachlorobicyclo[2.2.1]hept-5-ene-2,3-dicarboximide), himic acid N-hydroxyimide (N-hydroxy-bicyclo[2.2.1]hept-5-ene-2,3-dicarboximide, N-hydroxytrimellitimide, N,N′-dihydroxypyromellitic diimide, N,N′-dihydroxynaphthalenetetracarboxylic diimide, α,β-diacetoxy-N-hydroxysuccinimide, N-hydroxy-α,β-bis(propionyloxy)succinimide, N-hydroxy-α,β-bis(valeryloxy)succinimide, N-hydroxy-α,β-bis(lauroyloxy)succinimide, α,β-bis(benzoyloxy)-N-hydroxysuccinimide, N-hydroxy-4-methoxycarbonylphthalimide, 4-ethoxycarbonyl-N-hydroxyphthalimide, N-hydroxy-4-pentyloxycarbonylphthalimide, 4-dodecyloxy-N-hydroxycarbonylphthalimide, N-hydroxy-4-phenoxycarbonylphthalimide, N-hydroxy-4,5-bis(methoxycarbonyl)phthalimide, 4,5-bis(ethoxycarbonyl)-N-hydroxyphthalimide, N-hydroxy-4,5-bis(pentyloxycarbonyl)phthalimide, 4,5-bis(dodecyloxycarbonyl)-N-hydroxyphthalimide, and N-hydroxy-4,5-bis(phenoxycarbonyl)phthalimide; compounds corresponding to these compounds, except with R being acyl such as acetyl, propionyl, or benzoyl; compounds of Formula (1) in which X is an —OR group, and R is a group capable of forming an acetal or hemiacetal bond with hydroxy, such as N-methoxymethyloxyphthalimide, N-(2-methoxyethoxymethyloxy)phthalimide, and N-tetrahydropyranyloxyphthalimide; compounds of Formula (1) in which X is an —OR group, and R is sulfonyl, such as N-methanesulfonyloxyphthalimide and N-(p-toluenesulfonyloxy)phthalimide; and compounds of Formula (1), in which X is an —OR group, and R is a group resulting from removing an OH group from an inorganic acid, such as sulfuric ester, nitric ester, phosphoric ester, and boric ester of N-hydroxyphthalimide.

Of the preferred imide compounds, representative, but non-limiting examples of compounds having a 6-membered cyclic imide skeleton include compounds of Formula (1) in which X is an —OR group, and R is hydrogen, such as N-hydroxyglutarimide, N-hydroxy-α,α-dimethylglutarimide, N-hydroxy-β,β-dimethylglutarimide, N-hydroxy-1,8-decahydronaphthalenedicarboximide, N,N′-dihydroxy-1,8;4,5-decahydronaphthalenetetracarboxylic diimide, N-hydroxy-1,8-naphthalenedicarboximide (N-hydroxynaphthalimide), and N,N′-dihydroxy-1,8;4,5-naphthalenetetracarboxylic diimide; compounds corresponding to these compounds, except with R being acyl such as acetyl, propionyl, or benzoyl; compounds of Formula (1) in which X is an —OR group, and R is a group capable of forming an acetal or hemiacetal bond with hydroxy, such as N-methoxymethyloxy-1,8-naphthalenedicarboximide and N,N′-bis(methoxymethyloxy)-1,8;4,5-naphthalenetetracarboxylic diimide; compounds of Formula (1) in which X is an —OR group, and R is sulfonyl, such as N-methanesulfonyloxy-1,8-naphthalenedicarboximide and N,N′-bis(methanesulfonyloxy)-1,8;4,5-naphthalenetetracarboxylic diimide; and compounds of Formula (1) in which X is an —OR group, and R is a group resulting from removing an OH group from an inorganic acid, such as sulfuric ester, nitric ester, phosphoric ester, and boric ester of N-hydroxy-1,8-naphthalenedicarboximide or of N,N′-dihydroxy-1,8;4,5-naphthalenetetracarboxylic diimide.

Of the imide compounds, N-hydroxyimide compounds (compounds where X is an —OR group, and R is hydrogen) can each be produced typically by common imidization reactions (reaction methods) such as a method in which a corresponding acid anhydride is imidized by allowing the same to react with hydroxylamine and to undergo ring-opening and ring-closing of the acid anhydride group. Of the imide compounds, compounds where X is an —OR group, and R is a hydroxy-protecting group can be produced typically by introducing a desired protecting group or groups into corresponding compounds (N-hydroxyimide compounds) where R is hydrogen, using common reactions for introducing protecting groups. For example, N-acetoxyphthalimide can be produced typically by allowing N-hydroxyphthalimide to react with acetic anhydride, or to react with an acetyl halide in the presence of a base.

Particularly preferred, but non-limiting examples of the imide compounds include N-hydroxyimide compounds derived from aliphatic multivalent carboxylic anhydrides or aromatic multivalent carboxylic anhydrides, such as N-hydroxysuccinimide (SP: 33.5 (MPa)^{1/2}), N-hydroxyphthalimide (SP: 33.4 (MPa)^{1/2}), N,N′-dihydroxypyromellitic diimide, N-hydroxyglutarimide, N-hydroxy-1,8-naphthalenedicarboximide, and N,N′-dihydroxy-1,8;4,5-naphthalenetetracarboxylic diimide; and compounds resulting from introducing a protecting group or groups into hydroxy of the N-hydroxyimide compounds.

Each of different imide compounds may be used alone or in combination. The imide compound or compounds may be formed in the reaction system. However, commercial products available typically under the trade names of N-Hydroxyphthalimide (from Wako Pure Chemical Industries, Ltd.); and N-Hydroxysuccinimide (from Wako Pure Chemical Industries, Ltd.) can be advantageously used in the present invention.

Upon use, the imide compounds may also be supported on supports (exemplified by porous supports such as activated carbon, zeolite, silica, silica-alumina, and bentonite.

The imide compound or compounds may be used in an amount of typically about 0.0000001 to about 1 mole, preferably 0.00001 to 0.5 mole, and particularly preferably 0.0001 to 0.4 mole, per mole of the substrate (A). The imide compound or compounds, when used in an amount within the range, allow the oxidation reaction to proceed at a high reaction rate.

Metal Compound

The present invention may employ a metal compound or compounds (excluding those corresponding to the solid acid catalyst) in addition to the solid acid catalyst, for still higher reaction rate.

Non-limiting examples of metal elements constituting the metal compounds include cobalt, manganese, zirconium, and molybdenum. The metal elements may each have any valence not limited and may have a valence of typically 0 to about 6.

Non-limiting examples of the metal compounds include, of the metal elements, inorganic compounds and organic compounds. Non-limiting examples of the inorganic compounds include elementary substances, hydroxides, oxides (including complex oxides), halides (fluorides, chlorides, bromides, and iodides), oxoacid salts (such as nitrates, sulfates, phosphates, borates, and carbonates), isopolyacid salts, and heteropolyacid salts. Non-limiting examples of the organic compounds include organic acid salts such as acetates, propionates, cyanides, naphthenates, and stearates; and complexes.

Non-limiting examples of ligands constituting the complexes include OH (hydroxo), alkoxys (such as methoxy, ethoxy, propoxy, and butoxy), acyls (such as acetyl and propionyl), alkoxycarbonyls (such as methoxycarbonyl and ethoxycarbonyl), acetylacetonato, cyclopentadienyl, halogen atoms (such as chlorine and bromine), CO, CN, oxygen atom, H₂O (aquo), phosphorus compounds exemplified by phosphines (such as triphenylphosphine and other triarylphosphines), and nitrogen-containing compounds such as NH₃ (ammine), NO, NO₂ (nitro), NO₃ (nitrato), ethylenediamine, diethylenetriamine, pyridine, and phenanthroline.

Specific, but non-limiting examples of the metal compounds include, when taking cobalt compounds as an example, divalent or trivalent cobalt compounds exemplified by inorganic compounds such as cobalt hydroxides, cobalt oxides, cobalt chlorides, cobalt bromides, cobalt nitrates, cobalt sulfates, and cobalt phosphate; organic acid salts such as cobalt acetate, cobalt naphthenate, and cobalt stearate; and complexes such as acetylacetonatocobalt. Non-limiting examples of compounds of other metal elements include compounds corresponding to the cobalt compounds. Each of different metal compounds may be used alone or in combination. For significantly higher reaction rates in the present invention, at least a cobalt compound is preferably used and is particularly preferably used in combination with a manganese compound.

The metal compound may be used in an amount of typically about 0.00001 to about 10 mole percent, and preferably 0.2 to 2 mole percent, relative to the substrate (A). When two or more different metal compounds are used, the term “amount” refers to the total amount of them. When the imide compound is used, the metal compound may be used in an amount of typically about 0.001 to about 10 moles, preferably 0.005 to 3 moles, and particularly preferably 0.01 to 1 moles, per mole of the imide compound.

Oxidation Reaction

The reaction temperature in the oxidation reaction in the present invention can be selected as appropriate according typically to the types of the substrate (A) and the target product and is typically around room temperature to around 200° C., preferably 50° C. to 130° C., and particularly preferably 60° C. to 100° C. The reaction can be performed at normal atmospheric pressure or under pressure (under a load). When the reaction is performed under pressure (under a load), the reaction pressure is generally about 0.1 to about 10 MPa, preferably 0.15 to 8 MPa, and particularly preferably 0.5 to 8 MPa. The present invention allows the oxidation reaction to proceed smoothly even at normal atmospheric pressure (0.1 MPa), because of using the oxidizer oxygen in combination with ozone.

The reaction time can be adjusted as appropriate according to the reaction temperature and pressure and is typically about 0.1 to about 20 hours, and preferably 1 to 10 hours.

The oxidation reaction can be performed according to a common system or procedure such as a batch system, semi-batch system, or continuous system. When the imide compound is used, a gradual (continuous or intermittent) addition of the imide compound may contribute to oxidation of the substrate with a higher conversion to give an oxide at a higher selectivity.

The reaction gives an oxide or oxides (oxidation product or products) corresponding to the substrate (A) used in the reaction. Non-limiting examples of such oxides include alcohols, ketones, aldehydes, carboxylic acids, and hydroperoxides. The heteroatom-containing compounds (A1) containing a carbon-hydrogen bond at a position adjacent to the heteroatom, when used as the substrate (A), tend to give compounds in which a carbon atom at a position adjacent to the heteroatom is oxidized. The compounds (A2) containing a carbon-heteroatom double bond (such as carbonyl-containing compounds), when used, tend to give compounds in which the carbon atom (such as a carbonyl-carbon atom) involved in the carbon-heteroatom double bond is oxidized. The compounds (A3) containing a methine carbon atom, when used, tend to give compounds in which the methine carbon atom is oxidized. The compounds (A4) containing an unsaturated bond and a carbon-hydrogen bond at a position adjacent to the unsaturated bond, when used, tend to give compounds in which the carbon atom involved in the carbon-hydrogen bond is oxidized. The alicyclic compounds (A5), when used, tend to give compounds in which a carbon atom constituting the alicyclic ring is oxidized. The conjugated compounds (A6), when used, tend to give compounds in which a carbon atom constituting the conjugated double bond is oxidized. The amine compounds (A7), when used, tend to give compounds in which a nitrogen atom constituting the amino group, or a carbon atom at a position adjacent to the nitrogen atom is oxidized. The aromatic compounds (A8), when used, readily allow a carbon atom constituting the aromatic ring to be oxidized. When a hydrocarbon group is bonded to the aromatic ring, the aromatic compounds (A8) tend to give compounds in which a carbon atom at a position adjacent to the aromatic ring is oxidized. When the straight chain alkanes (A9) are used, a terminal carbon atom tends to be oxidized, but a methylene carbon atom may be oxidized. The olefins (A10), when used, tend to give compounds in which a carbon atom involved in the carbon-carbon double bond, or a carbon atom at a position adjacent to the carbon atom is oxidized. The olefins (A10) may give epoxy compounds.

After the completion of the reaction, the oxide or oxides can be separated/purified by a separation process such as filtration, concentration, distillation, extraction, crystallization, recrystallization, or column chromatography, or a separation process as any combination of them.

The method according to the present invention can rapidly oxidize the substrates (A) (even hardly-oxidizable substrates such as straight chain alkanes) under mild conditions to efficiently produce corresponding oxides (such as oxygen-containing compounds). In addition, assume that an imide compound having a cyclic imide skeleton is used so as to offer higher reaction rates. In this case, the method allows the oxidation reaction to proceed rapidly even using approximately no solvent, even when the imide compound has low solubility (such as one having a solubility parameter SP greater than 26) with respect to the substrates (such as hydrocarbons).

### EXAMPLES

The present invention will be illustrated in further detail with reference to several examples below. It should be noted, however, that the examples are by no means intended to limit the scope of the present invention.

### Example 1

In a 30-mL three-necked flask, 0.300 g (0.071 g in terms of cobalt) of cobalt acetate tetrahydrate and 0.293 g (0.066 g in terms of manganese) of manganese acetate tetrahydrate were placed, and combined with 10.0 g of acetic acid to give a homogeneous solution. The solution was combined with 3.00 g of Nafion pellets (trade name Nafion, supplied by E. I. du Pont de Nemours & Co., a fluorinated sulfonic acid resin), followed by stirring at 100° C. for 8 hours. The solids were filtrated and washed with 30 mL of acetic acid. Next, the solids were sequentially washed with 30 mL of ethyl acetate and 30 mL of diethyl ether, were dried at 130° C. under reduced pressure for 5 hours, and yielded 3.17 g of Nafion pellets supporting cobalt and manganese.

The above-prepared Nafion pellets supporting cobalt and manganese were subjected to the following operation so as to determine the amounts of supported cobalt and manganese.

Specifically, a small amount of the Nafion pellets supporting cobalt and manganese was sampled, immersed in a 10 weight percent dilute nitric acid aqueous solution and left stand overnight at room temperature to elute or dissolve all the supported metals into the aqueous phase.

The metal concentrations in the aqueous phase were analyzed by ICP, on the basis of which the amounts of cobalt and manganese as supported on the Nafion pellets were calculated and found that cobalt and manganese were supported in amounts respectively of 2.0% by weight and 1.9% by weight.

### Example 2

A 20 weight percent Nafion dispersion (supplied by E. I. du Pont de Nemours & Co.) was allowed to flow through within a Teflon® tube having an inside diameter of 2.0 mm and a length of 1.5 m, and the resulting article was dried at about 100° C. and yielded a Teflon® tube bearing, on its inner wall, a 0.5-mm thick Nafion layer. The resulting tube had an inside diameter of 1.0 mm. Through the tube, a 5 weight percent solution of cobalt acetate (Co(OAc)₂) in acetic acid was allowed to flow, the resulting article was heated at 100° C. for one hour, was dried at 120° C., and yielded a flow reactor bearing, on its inner wall, a layer or coating of a super acidic ion exchange resin (fluorinated sulfonic acid resin) being ion-exchanged with cobalt ions. The cobalt concentration in the super acidic ion exchange resin coating was measured by inductively coupled plasma (ICP) emission spectrometry and was found to be 2.4 weight percent.

### Example 3

In a 100-mL steel-use-stainless (SUS) pressure-tight reactor (supplied by Taiatsu Techno Corporation, Model TVS-1) equipped with a gas flow line and an insertion tube, 0.808 g (4.95 mmol, 10.0 mole percent relative to the substrate) of N-hydroxyphthalimide (hereinafter also referred to as “NHPI”, SP: 33.4 (MPa)^{1/2}) and 10.0 g (50.4 mmol) of tetradecane (supplied by Tokyo Chemical Industry Co., Ltd.) were charged, and the reactor was set on an oil bath, and the gas flow line was coupled. Air containing ozone gas and having an oxygen content of 20.7 volume percent and an ozone gas content of 0.3 volume percent was prepared. The ozone gas had been generated using an ozone generator (trade name SG-01-PSA2, supplied by Sumitomo Precision Products Co., Ltd.). Bubbling of the ozone-gas-containing air into the liquid in the reactor was started at a rate of 1000 mL/min, and heating of the reactor was started. At the time point when the internal temperature reached 90° C., 0.635 g of the Nafion pellets supporting cobalt and manganese, prepared in Example 1, was added to start a reaction, where the Nafion pellets supported cobalt and manganese in amounts respectively of 2.0% by weight and 1.9% by weight. Eight hours into the reaction at 90° C., the conversion from tetradecane was measured by gas chromatography (column: 007-FFAP) and was found to be 4.9%, where tetradecanol, tetradecanone, and tetradecanoic acid were formed.

### Comparative Example 1

A procedure as in Example 3 was performed, except for using, instead of the ozone-gas-added air, air without addition of the ozone gas. As a result, the substrate conversion was found to be 0.4%.

### Example 4

A procedure as in Example 3 was performed, except for not using NHPI. As a result, the substrate conversion was found to be 2.9%.

### Example 5

A procedure as in Example 3 was performed, except for using 2,2,4-trimethylpentane as a substrate instead of tetradecane. As a result, the substrate conversion was found to be 6.4%, where 4-hydroxy-2,2,4-trimethylpentane, neopentyl alcohol, and acetone were formed.

### Comparative Example 2

A procedure as in Example 5 was performed, except for using, instead of the ozone-gas-added air, air without addition of the ozone gas. As a result, the substrate conversion was found to be 0.5%.

### Example 6

A procedure as in Example 3 was performed, except for using n-heptane as a substrate instead of tetradecane. As a result, the substrate conversion was found to be 4.9%, where 2-hydroxyheptane, 2-heptanone, and 2,6-heptanedione were formed.

### Comparative Example 3

A procedure as in Example 6 was performed, except for using, instead of the ozone-gas-added air, air without addition of the ozone gas. As a result, the substrate conversion was found to be 0.4%.

### Example 7

A procedure as in Example 3 was performed, except for using toluene as a substrate instead of tetradecane. As a result, the substrate conversion was found to be 33%, where benzyl alcohol, benzaldehyde, and benzoic acid were formed.

### Comparative Example 4

A procedure as in Example 7 was performed, except for using, instead of the ozone-gas-added air, air without addition of the ozone gas. As a result, the substrate conversion was found to be 2.6%.

### Example 8

A procedure as in Example 3 was performed, except for using cyclohexane (833 mmol) as a substrate instead of tetradecane. As a result, the substrate conversion was found to be 8%, where cyclohexanone and cyclohexanol were formed.

### Comparative Example 5

A procedure as in Example 8 was performed, except for using, instead of the ozone-gas-added air, air without addition of the ozone gas. As a result, the substrate conversion was found to be 1.0%.

### Example 9

A procedure as in Example 3 was performed, except for using cyclohexene (889 mmol) as a substrate instead of tetradecane. As a result, the substrate conversion was found to be 10%, where 2,3-cyclohexen-1-ol and 2,3-cyclohexen-1-one were formed.

### Comparative Example 6

A procedure as in Example 9 was performed, except for using, instead of the ozone-gas-added air, air without addition of the ozone gas. As a result, the substrate conversion was found to be 1.3%.

### Example 10

A procedure as in Example 3 was performed, except for using isochroman (716 mmol) as a substrate instead of tetradecane. As a result, the substrate conversion was found to be 14%, where 3,4-dihydroisocoumalin was formed.

### Comparative Example 7

A procedure as in Example 10 was performed, except for using, instead of the ozone-gas-added air, air without addition of the ozone gas. As a result, the substrate conversion was found to be 1.7%.

### Example 11

A procedure as in Example 3 was performed, except for using cumene (647 mmol) as a substrate instead of tetradecane, and for adding sulfuric acid after the completion of the reaction. As a result, the substrate conversion was found to be 11%, where phenol was formed.

### Comparative Example 8

A procedure as in Example 11 was performed, except for using, instead of the ozone-gas-added air, air without addition of the ozone gas. As a result, the substrate conversion was found to be 1.5%.

### INDUSTRIAL APPLICABILITY

According to the present invention, a substrate is oxidized in the presence of the specific solid acid catalyst and in the coexistence of oxygen and ozone. This configuration offers significantly higher oxidation rates and enables oxidation of a wide variety of substrates with high conversions. In addition, the configuration gives an oxide (such as an oxygen-containing compound) in a high yield even when the substrate is selected from hydrocarbons, in particular, selected from straight chain alkanes.

1. A solid acid catalyst for use in an oxidation reaction to oxidize a substrate (A) in coexistence of oxygen and ozone, the solid acid catalyst comprising:
a transition metal in a form of an elementary substance, a compound, or an ion; and
a support supporting the transition metal, the support comprising, at least on a surface thereof,
a strong acid or super strong acid having a Hammett acidity function (H₀) of −9 or less,
the substrate (A) comprising a compound selected from the group consisting of:
(A1) compounds containing a heteroatom and a carbon-hydrogen bond at a position adjacent to the heteroatom;
(A2) compounds containing a carbon-heteroatom double bond;
(A3) compounds containing a methine carbon atom;
(A4) compounds containing an unsaturated bond and a carbon-hydrogen bond at a position adjacent to the unsaturated bond;
(A5) alicyclic compounds;
(A6) conjugated compounds;
(A7) amine compounds;
(A8) aromatic compounds;
(A9) straight chain alkanes; and
(A10) olefins.

2. A solid acid catalyst for use in an oxidation reaction to oxidize a substrate (A) in coexistence of oxygen and ozone, the solid acid catalyst comprising:
a transition metal in a form of an elementary substance, a compound, or an ion; and
a support supporting the transition metal, the support comprising, at least on a surface thereof, at least one strong acid or super strong acid selected from the group consisting of:
sulfuric acid;
sulfated metal oxides;
noble metals/sulfated metal oxides;
metal oxide super strong acids; and
fluorinated sulfonic acid resins,
the substrate (A) comprising a compound selected from the group consisting of:
(A1) compounds containing a heteroatom and a carbon-hydrogen bond at a position adjacent to the heteroatom;
(A2) compounds containing a carbon-heteroatom double bond;
(A3) compounds containing a methine carbon atom;
(A4) compounds containing an unsaturated bond and a carbon-hydrogen bond at a position adjacent to the unsaturated bond;
(A5) alicyclic compounds;
(A6) conjugated compounds;
(A7) amine compounds;
(A8) aromatic compounds;
(A9) straight chain alkanes; and
(A10) olefins.

3. The solid acid catalyst according to claim 1,
wherein the substrate (A) is selected from hydrocarbons.

4. The solid acid catalyst according to claim 1,
wherein the support is selected from:
a support comprising
a fluorinated sulfonic acid resin in a form of a pellet or a particle; and
a support comprising:
a solid; and
a fluorinated sulfonic acid resin in a form of a layer disposed on the solid.

5. The solid acid catalyst according to claim 1,
wherein the transition metal is at least one selected from the group consisting of cobalt, manganese, vanadium, iron, zirconium, tungsten, and molybdenum.

6. The solid acid catalyst according to claim 1,
which is pellet-like, film-like, or tubular in shape.

7. A method for producing an oxide, the method comprising
oxidizing a substrate (A) in coexistence of oxygen and ozone and in presence of the solid acid catalyst according to claim 1,
the substrate (A) comprising a compound selected from the group consisting of:
(A1) compounds containing a heteroatom and a carbon-hydrogen bond at a position adjacent to the heteroatom;
(A2) compounds containing a carbon-heteroatom double bond;
(A3) compounds containing a methine carbon atom;
(A4) compounds containing an unsaturated bond and a carbon-hydrogen bond at a position adjacent to the unsaturated bond;
(A5) alicyclic compounds;
(A6) conjugated compounds;
(A7) amine compounds;
(A8) aromatic compounds;
(A9) straight chain alkanes; and
(A10) olefins.

8. The method for producing an oxide,
wherein the substrate is oxidized further in presence of an imide compound having a cyclic imide skeleton.

9. The solid acid catalyst according to claim 2,
wherein the substrate (A) is selected from hydrocarbons.

10. The solid acid catalyst according to claim 2,
wherein the support is selected from:
a support comprising
a fluorinated sulfonic acid resin in a form of a pellet or a particle; and
a support comprising:
a solid; and
a fluorinated sulfonic acid resin in a form of a layer disposed on the solid.

11. The solid acid catalyst according to claim 3,
wherein the support is selected from:
a support comprising
a fluorinated sulfonic acid resin in a form of a pellet or a particle; and
a support comprising:
a solid; and
a fluorinated sulfonic acid resin in a form of a layer disposed on the solid.

12. The solid acid catalyst according to claim 2,
wherein the transition metal is at least one selected from the group consisting of cobalt, manganese, vanadium, iron, zirconium, tungsten, and molybdenum.

13. The solid acid catalyst according to claim 3,
wherein the transition metal is at least one selected from the group consisting of cobalt, manganese, vanadium, iron, zirconium, tungsten, and molybdenum.

14. The solid acid catalyst according to claim 4,
wherein the transition metal is at least one selected from the group consisting of cobalt, manganese, vanadium, iron, zirconium, tungsten, and molybdenum.

15. The solid acid catalyst according to claim 2,
which is pellet-like, film-like, or tubular in shape.

16. The solid acid catalyst according to claim 3,
which is pellet-like, film-like, or tubular in shape.

17. The solid acid catalyst according to claim 4,
which is pellet-like, film-like, or tubular in shape.

18. The solid acid catalyst according to claim 5,
which is pellet-like, film-like, or tubular in shape.

19. A method for producing an oxide, the method comprising
oxidizing a substrate (A) in coexistence of oxygen and ozone and in presence of the solid acid catalyst according to claim 2,
the substrate (A) comprising a compound selected from the group consisting of:
(A1) compounds containing a heteroatom and a carbon-hydrogen bond at a position adjacent to the heteroatom;
(A2) compounds containing a carbon-heteroatom double bond;
(A3) compounds containing a methine carbon atom;
(A4) compounds containing an unsaturated bond and a carbon-hydrogen bond at a position adjacent to the unsaturated bond;
(A5) alicyclic compounds;
(A6) conjugated compounds;
(A7) amine compounds;
(A8) aromatic compounds;
(A9) straight chain alkanes; and
(A10) olefins.

20. A method for producing an oxide, the method comprising
oxidizing a substrate (A) in coexistence of oxygen and ozone and in presence of the solid acid catalyst according to claim 3,
the substrate (A) comprising a compound selected from the group consisting of:
(A1) compounds containing a heteroatom and a carbon-hydrogen bond at a position adjacent to the heteroatom;
(A2) compounds containing a carbon-heteroatom double bond;
(A3) compounds containing a methine carbon atom;
(A4) compounds containing an unsaturated bond and a carbon-hydrogen bond at a position adjacent to the unsaturated bond;
(A5) alicyclic compounds;
(A6) conjugated compounds;
(A7) amine compounds;
(A8) aromatic compounds;
(A9) straight chain alkanes; and
(A10) olefins.
